(19) United States
(12) Patent Application Publication
(10) Pub. No.: US 2012/0172785 A1
(43) Pub. Date: Jul. 5, 2012

(54) CASSETTE PRISM FOR FLUID LEVEL DETECTION

(76) Inventors: Richard A. Belley, St. Louis, MO (US); Harrell Keith Nation, Freeburg, IL (US)

(21) Appl. No.: 12/980,756

(22) Filed: Dec. 29, 2010

(51) Int. Cl. A61F 9/007

(52) U.S. Cl. 604/22

(57) ABSTRACT

A surgical cassette has rigid walls for collecting aspirated tissue and fluid and for operable communication with a fluid level detection system of a surgical console, in which the surgical cassette is placed. The surgical cassette has an interior volume, defined by the rigid walls, to hold the aspirated tissue and fluid. A plurality of prisms are formed on an interior of at least one rigid wall, at a location, such that the prisms are in operable communication with the fluid level detection system, when the surgical cassette is placed in the surgical console. An exterior of the rigid wall corresponding to the location of the plurality of prisms is coplanar with an exterior of the rigid wall not corresponding to the location of the plurality of prisms.

### FIELD

The present embodiment relates to fluid collection cassettes for surgery and, more particularly, to surgical cassettes for collecting aspirated tissue and fluid.

### BACKGROUND

This section provides background information related to the present disclosure which is not necessarily prior art.

Surgical cassettes for collecting aspirated fluid and tissue from a surgical instrument, such as a phacoemulsification handpiece or a vitreous cutter, are well-known. It is also known to provide a fluid level detection scheme for a surgical console to determine some level of fluid in the cassette. Known level detection schemes provide a wide variation of detail regarding the fluid level that is detected. Some known level detection schemes only provide a user with a notice that the collection cassette is full or near full, while others provide a running or periodic notice of a measured or estimated amount of fluid and tissue in the cassette throughout surgery.

There are many schemes known in the art to detect a fluid level in the cassette. Some examples include floating ball or rocker arms that will rise as the fluid level rises and, when the fluid level is full or near full, the ball or arm may disrupt an optical circuit path triggering a warning to a user or stopping aspiration until the cassette is emptied. It is also known to provide spaced electrodes in the cassette, such that when aspirated fluid contacts the electrodes a circuit path is completed and a warning is triggered.

Another scheme for fluid level detection is described in the published U.S. patent application 2007/0287959 by Walter et al., assigned to Bausch & Lomb Incorporated, and entitled Ophthalmic Surgical Cassette and System. This application teaches the use of a fluid level indicator formed as a notched section in a wall of a collection cassette. The notched section cooperates with a photo-detector to measure a fluid level based on the difference in refraction and reflection of light in parts of the notched section, corresponding to a fluid level in the cassette compared to the refraction and reflection of light in parts of the notched section above the fluid level in the cassette. While this fluid level detection scheme is accurate and reliable when manufactured to relatively tight tolerances, it would be desirable to provide a more robust fluid level indicator that is less reliant on precise alignment between the fluid level indicator and the photo-detector.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a perspective view of a prior art surgical cassette;

FIG. 2 is a partial cross-section of FIG. 1 along line 2-2;

FIG. 3 is a partial cross-section of a surgical cassette, in accordance with a present exemplary embodiment;

FIG. 4 is a partial perspective view of the surgical cassette of FIG. 3;

FIG. 5 is a partial perspective view of another surgical cassette, in accordance with a present exemplary embodiment;

FIG. 6 is a partial perspective view of yet another surgical cassette, in accordance with a present exemplary embodiment; and

FIG. 7 is a partial perspective view of still another surgical cassette, in accordance with a present exemplary embodiment.

Corresponding reference numerals indicate corresponding parts throughout the several views of the drawings.

### DETAILED DESCRIPTION

Example embodiments will now be described more fully with reference to the accompanying drawings.

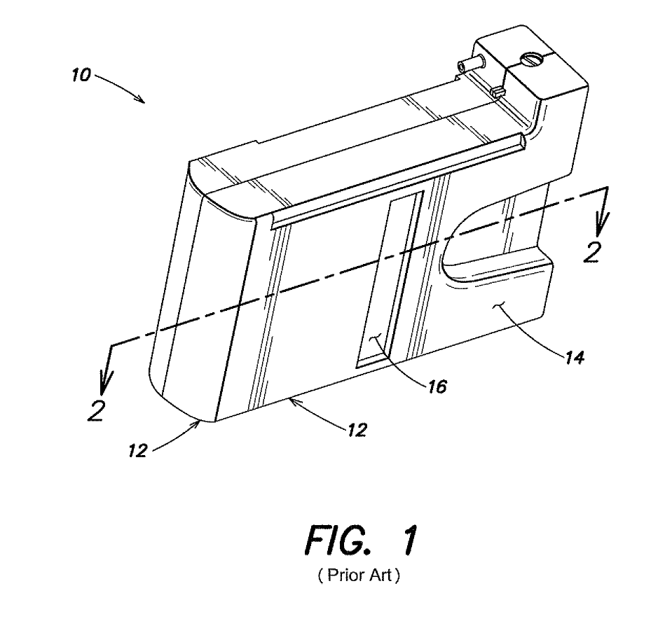

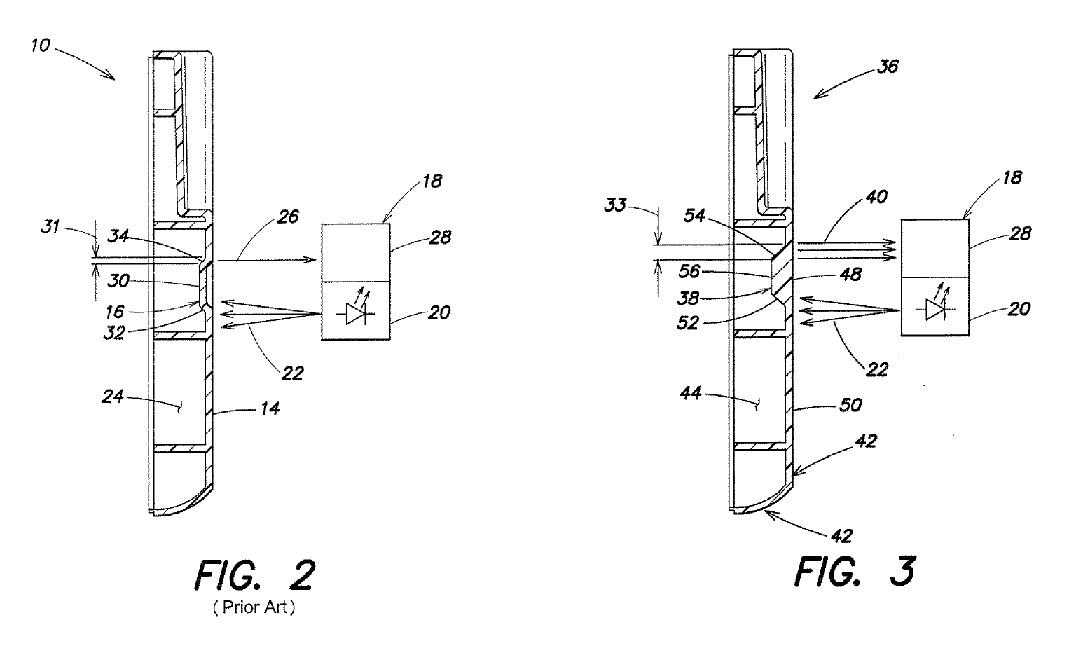

FIG. 1 is a prior art perspective view of a portion of a cassette 10. Cassette 10 and its interaction with a photo-detector are explained in detail in the above cited U.S. patent publication 2007/0287959, which is incorporated by reference in this disclosure. Cassette 10 is formed by rigid walls 12 and has an interior volume to collect aspirated tissue and fluid. One of the rigid walls 12 has an exterior 14 with a notched section 16 recessed from the exterior 14, as best seen in FIG. 2.

A fluid level detection system or photo-detector 18 (both terms are used interchangeably), that is part of a surgical console not shown, is also seen in FIG. 2 to show the interaction between the notched section 16 and the photo-detector 18. Fluid level detection system 18 includes a light source 20 that projects light onto the notched section 16, as shown by arrows 22. Some light from source 20 is then refracted into an interior of cassette 10 and some light is reflected back to photo-detector 18 (shown as arrow 26) and is received by sensor 28. The amount of light received by sensor 28 will be sufficiently different between areas of the notched section, corresponding to a fluid level of the cassette and to areas corresponding to portions of the notched section above the cassette fluid level that the sensor 28 can send a signal indicating the level of aspirated fluid to a processor (not shown) of a surgical console (not shown).

Notched section 16, as shown, has a face section 30 essentially parallel to the exterior 14 and a pair of angled side sections 32 and 34 connecting the face section 30 to the wall 12, such that a prism is formed so that the fluid level of the cassette 10 can be determined by the photo-detector 18.

Photo-detector 18 could be an appropriate imaging device and light source, such as charge coupled devices (CCD) or CMOS devices or preferably could be a contact image sensor (CIS). A CIS is essentially a one-dimensional array of photo-detectors used to create images. One example of a contact image sensor is Model M106-A6-R1 module available from CMOS Sensors Inc. Such contact image sensors are relatively small in size and are advantageous for use in the present invention. Though as stated above, other photo-detectors may be used in accordance with the present invention. The light source 20 can be a vertically oriented row of several light emitting diodes and are preferably within the red spectrum of light.

Notched section 16 and photo-detector 18 cooperate to detect a fluid level through the physics of light transmission through a prism and between boundaries of materials having different indexes of refraction. As those skilled in the art will appreciate, when light intersects a boundary between two mediums at a right angle, almost all of the light is transmitted through the boundary. However, when light intersects a boundary between two mediums at an angle of less than 90°, some of the light is transmitted and some of the light is reflected. Both the angle of the light and the change in the index of refraction between the two mediums determines how much of the light is transmitted and how much of the light is reflected. This principle is applied to the notched section to detect the fluid level in the cassette 10. Detecting the fluid level in cassette 10 with photo-detector 18 includes two sets of boundary conditions. One set of boundary conditions is present below the fluid level and another set of boundary conditions exist above the fluid level. Both sets of boundary conditions have two interfaces. One interface is between air and the cassette material and the other is between the cassette material and the contents of the cassette, i.e., aspirant fluid and tissue or air. The first interface, between air and cassette material, is insignificant since the amount of light reflected will be the same independent of the contents of the cassette. The second interface, between the cassette material and the cassette contents, is of most importance, since the amount of reflective light is directly related to the contents of the cassette.

Taking the reflected intensity concept described above one step further, by tailoring the reflection and the transmission coefficients, a greater difference between the fluid present and the fluid not present intensity can be achieved. This is achieved in cassette 10 by molding notched section 16 into the cassette wall. The amount of light received by the sensor 28 is directly proportional to the amount of light reflected at the medium boundaries. By monitoring the amount of light received at the sensor 28, the fluid level can be determined. The fluid level will correspond to the point in which the intensity of the reflected light changes.

When a cassette is empty, the sensor 28 is illuminated evenly. As the fluid level in the cassette rises, some of the light is dispersed into the fluid. The sensor 28 detects dimmer light below the fluid level compared to the detected light above the fluid level. A fluid level signal is then sent to an unshown processor, where a fluid level may be displayed on a screen and the aspiration may be stopped when a full level is detected.

The present exemplary embodiment shown in FIG. 3 uses the same concept and the same photo-detector 18 described above. A novel surgical cassette 36, with a plurality of prisms 38 replacing prior art notched section 16, is used to reflect light from source 20 back to sensor 28. As shown by arrows 40, it has been found that each prism 38 reflects more light to sensor 28 than was achieved with notched section 16. This increase in light reflection enhances the ability of sensor 28 to detect the fluid level of cassette 36 and makes the photo-detector 18 less sensitive to alignment with cassette 36, compared to the sensitivity of photo-detector 18 to alignment with cassette 10.

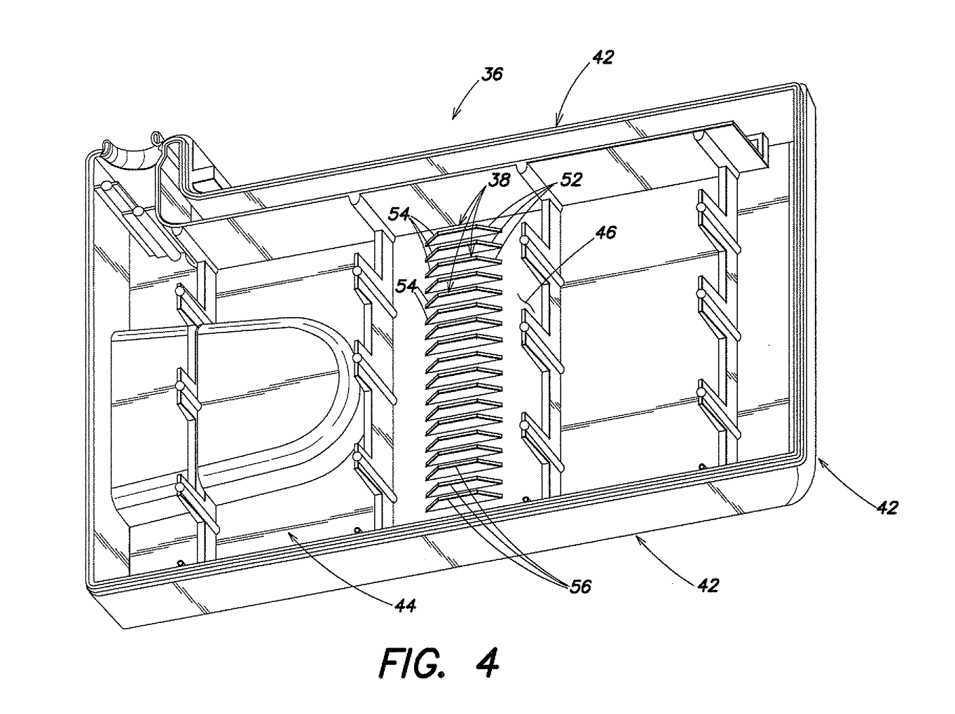

Surgical cassette 36, except for prisms 38, is identical to cassette 10 and has rigid walls 42 for collecting aspirated tissue and fluid, and is in operable communication with a fluid level detection system 18 (e.g. photo-detector) of a surgical console (not shown), in which the surgical cassette 36 is placed. The surgical cassette 36 has an interior volume 44 defined by the rigid walls 42, and holds the aspirated tissue and fluid. A plurality of prisms 38, best seen in FIG. 4, are formed on an interior 46 of at least one of the rigid walls 42. A location of the prisms 38 is such that the prisms 38 are in operable communication with the fluid level detection system 18 when the surgical cassette 36 is placed in the unshown surgical console. An exterior 48 of the rigid wall 42 corresponding to the location of the plurality of prisms 38 is coplanar with an exterior 50 of the rigid wall 42 not corresponding to the location of the plurality of prisms 38, as best seen in FIG. 3. Only a portion of cassette 36 is shown in FIGS. 3 and 4 for clarity. The cassette 36 may have the same dimensions and be formed of the same material as cassette 10. The only difference between cassette 36 and cassette 10 is that the prisms 38 replace the notched section 16.

A shape of each of the plurality of prisms 38 can be said to be trapezoidal. More particularly, the trapezoidal shape of the prisms 38 may be described as an isosceles trapezoid or a half-hexagon. As seen in FIGS. 3 and 4 each prism 38 has a pair of tapering side-walls 52 and 54 extending into the interior volume 44 from the rigid wall 42. The tapering side-walls 52 and 54 each preferably taper at an angle of forty-five degrees, with respect to the rigid wall 42. The tapering side-walls 52 and 54 are connected by a back-wall 56 that is generally parallel with rigid wall 42 connected to prisms 38.

The important performance difference between the notched section 16 of the prior art and the prisms 38, 62, and 70 of the present invention, is that a wider beam of light is transmitted from the cassettes of the present invention compared to the prior art cassette 10. The transmission of a wider beam is accomplished by providing longer tapering side-walls 52 and 54 that result in a wider surface to reflect light from prism 38 to fluid level detection system 18, as shown at 33, compared to the width of the reflective surface of notched section 16 shown at 31. Changing the width of the reflective surface alone may not be difficult on paper, in manufacturing a commercial product and attempting to produce a cassette with wider reflecting surfaces, it was found to be prohibitively expensive to produce such prisms over the required vertical length of the cassette. For example, a deeper notched section 16 could be made to provide wider side-walls 32 and 34. However to make such a part that is optically acceptable, requires molding the section 16 as one-piece and then welding it to one-half of cassette 10, and then welding the two halves of cassette 10 together—a very expensive process. In addition, attempting to manufacture a solid continuous prism with sufficiently wide reflective surfaces, resulted in molding sink marks and uneven material thickness that alter the optical properties of the material in places, and resulted in detection errors by sensor 28. It was discovered that molding several relatively thin prisms over the entire length, produced optically consistent material thickness, without sink marks, and at an acceptable cost. The wider beam directed toward sensor 28 by the present invention allows for greater tolerance variance in molding cassette 36 and for more variance in alignment between sensor 28 and cassette 36, without the need to change detector 18 or its associated software.

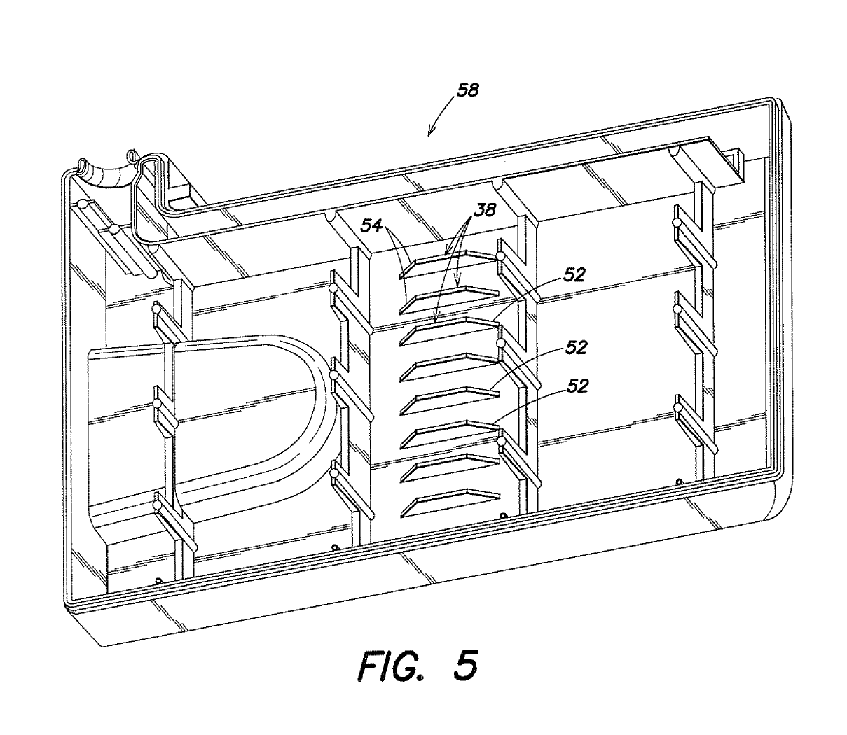

As those skilled in the art will appreciate, as the number of vertically aligned prisms 38 increases, the fluid level detection resolution that may be achieved by the detection system 18 will also increase. The number of prisms 38 may be at least three to provide a low level indicator, a level at which a warning that the cassette is becoming full may be generated, and a maximum level where a console may need to stop aspiration to prevent the cassette from over-flowing. Preferably, the number of prisms 38 is at least eight, as shown in cassette 58 of FIG. 5, but most preferably, the number of prisms 38 is at least 18, as shown in FIG. 4. The number of prisms that is most desirable is the number that allows the sensor and its associated software to receive reflected light in the same manner as it would with a single continuous prism.

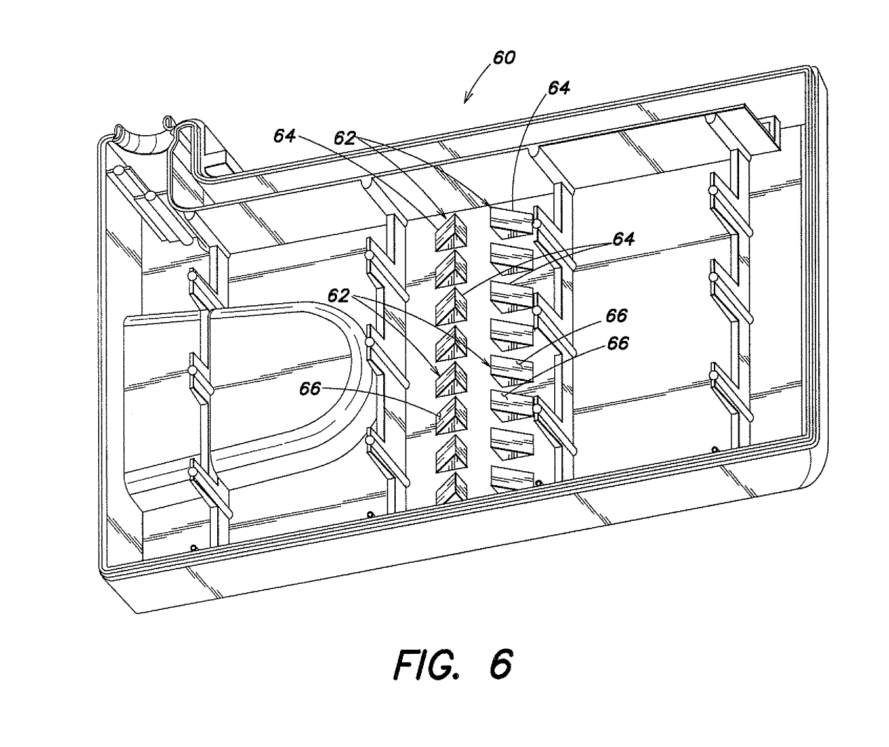

FIG. 6 shows a portion of another exemplary embodiment of a cassette 60, which is identical to cassette 36, except that each of the plurality of prisms 62 are formed by a pair of spaced-apart right-triangles 64. A hypotenuse 66 of each right-triangle 64 faces away from the other right-triangle 64 of the prism 62, and the pair of right-triangles 64 of each prism 62 are aligned vertically.

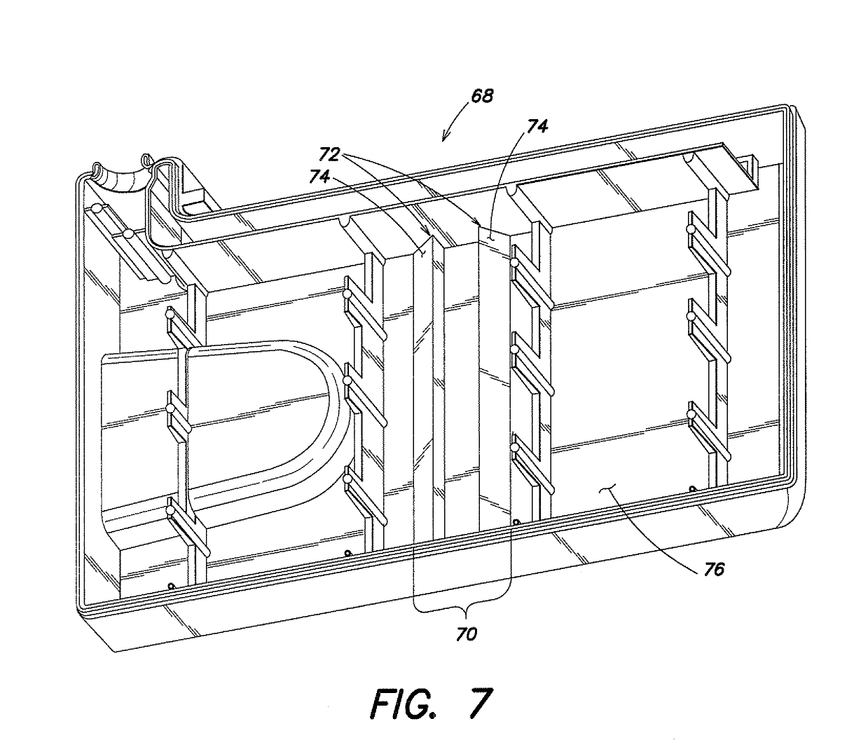

FIG. 7 shows a portion of still another exemplary embodiment of a cassette 68 and is identical to cassette 36, except only one prism 70 is formed in cassette 68. Prism 70 includes a pair of spaced-apart elongated members 72. Each member 72 has a cross-sectional shape of a right-triangle. A hypotenuse 74 of the right-triangle of each member 72 faces away from the other member 72 of the prism 70. The pair of elongated members 72 spans a majority of a vertical height of the rigid wall 76. The prism 70 reduces the molding problems identified above with respect to a single continuous prism, but is not as effective as prisms 38 and 62 above because prism 70 still requires a continuous structure over a long vertical length.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

We claim:

1. A surgical cassette having rigid walls for collecting aspirated tissue and fluid and for operable communication with a fluid level detection system of a surgical console in which the surgical cassette is placed, the surgical cassette comprising:
an interior volume, defined by the rigid walls, for holding the aspirated tissue and fluid; and
a plurality of prisms, formed on an interior of at least one of the rigid walls, at a location such that the prisms are in operable communication with the fluid level detection system when the surgical cassette is placed in the surgical console, wherein an exterior of the rigid wall corresponding to the location of the plurality of prisms is coplanar with an exterior of the rigid wall not corresponding to the location of the plurality of prisms.

2. The surgical cassette of claim 1, wherein a shape of each of the plurality of prisms is trapezoidal.

3. The surgical cassette of claim 2, wherein the trapezoidal shape of the prism is an isosceles trapezoid.

4. The surgical cassette of claim 2, wherein each prism has a pair of tapering side-walls extending into the interior volume from the rigid wall.

5. The surgical cassette of claim 4, wherein the tapering side-walls each taper at an angle of forty-five degrees with respect to the rigid wall.

6. The surgical cassette of claim 1, wherein each of the plurality of prisms are formed by a pair of spaced-apart right-triangles with a hypotenuse of each right-triangle facing away from the other right-triangle of the prism and where the pair of right-triangles of each prism are aligned vertically.

7. The surgical cassette of claim 1, wherein a number of prisms is at least 3.

8. The surgical cassette of claim 1, wherein a number of prisms is at least 8.

9. The surgical cassette of claim 1, wherein a number of prisms is at least 18.

10. The surgical cassette of claim 1 having only one prism including a pair of spaced-apart elongated members each having a cross-sectional shape of a right-triangle with a hypotenuse of the right-triangle of each member facing away from the other member of the prism pair and where the pair of elongated members spans a majority of a vertical height of the rigid wall.
